(19) United States
(12) Patent Application Publication
(10) Pub. No.: US 2016/0370333 A1
(43) Pub. Date: Dec. 22, 2016

(54) GENERATING FINE RESOLUTION AIR POLLUTION ESTIMATES

(71) Applicant: INTERNATIONAL BUSINESS MACHINES CORPORATION, Armonk, NY (US)

(72) Inventors: LI LI, BEIJING (CN); LIANG LIU, BEIJING (CN); JUN MEI QU, BEIJING (CN); CHAO ZHANG, BEIJING (CN); CHAO QIANG ZHU, BEIJING (CN)

(21) Appl. No.: 14/955,305

(22) Filed: Dec. 1, 2015

Related U.S. Application Data
(63) Continuation of application No. 14/745,766, filed on Jun. 22, 2015.

(51) Int. Cl. G01N 33/00

(52) U.S. Cl. CPC G01N 33/0004

(57) ABSTRACT

Embodiments include methods, systems and computer program products for storing graph data for a directed graph in a relational database. Aspects include receiving historic and real-time air quality data from a plurality of air quality measurement stations in a geographic area and dividing the geographic area into a plurality of locations. Aspects also include creating a composite air quality model for the geographic area based on a trend process model, a diffusion process model, a chemical process, and a containment process model based on the historic air quality data and creating a real-time air pollution estimate for each of the plurality of locations based on the composite air quality model and the real-time air quality data.

### DOMESTIC PRIORITY

This application is a Continuation application of legally related U.S. Ser. No. 14/745,766 filed Jun. 22, 2015, the contents of which in its entirety are herein incorporated by reference.

### BACKGROUND

The present disclosure relates to monitoring air pollution levels, and more specifically, to methods, systems and computer program products for calculating estimates of air pollution levels locations that do not have air quality measurement stations.

Air pollution is a worldwide problem and it is generally considered to be one of the biggest threats to human health. Air quality monitoring has been, and will continue to be, used to monitor and combat air pollution. However, the construction of air quality measurement stations is costly and the measurement stations require a lot of labor to install and maintain. As a result, in most cities that have air quality measurement stations the stations are sparely located. For example, in Beijing, a city covering approximately 10,000 km², there are only 35 air quality measurement stations. As a result, for locations that are not close to air quality measurement stations, air quality data is not available.

In general, air pollution can be modeled as a combination of several different processes, which include trend processes, diffusion processes, chemical processes, containment processes and the interactions between these processes. As used herein: a trend process is a process in which pollution is blown into an area; a diffusion process is a process in which pollution diffuses into an area; a chemical process is a process in which pollutants are formed in the air from a chemical change of gases, such when gases from burning fuels react with sunlight and water vapor; and a containment process is a process that reduces or restricts a trend process, a diffusion process and/or a chemical process. While methods have been developed for modeling air pollution based on trend processes, diffusion processes and/or chemical processes, none of the existing models take into consideration the existence or effect of containment processes of pollution levels.

### SUMMARY

In accordance with an embodiment, a method for generating fine resolution air pollution estimates is provided. The method includes receiving historic and real-time air quality data from a plurality of air quality measurement stations in a geographic area and dividing the geographic area into a plurality of locations. The method also includes creating, by a processor, a composite air quality model for the geographic area based on a trend process model, a diffusion process model, a chemical process, and a containment process model based on the historic air quality data and creating, by the processor, a real-time air pollution estimate for each of the plurality of locations based on the composite air quality model and the real-time air quality data.

In accordance with another embodiment, a processing system for generating fine resolution air pollution estimates includes a processor. The processor is configured to perform a method that includes receiving historic and real-time air quality data from a plurality of air quality measurement stations in a geographic area and dividing the geographic area into a plurality of locations. The method also includes creating a composite air quality model for the geographic area based on a trend process model, a diffusion process model, a chemical process, and a containment process model based on the historic air quality data and creating a real-time air pollution estimate for each of the plurality of locations based on the composite air quality model and the real-time air quality data.

In accordance with a further embodiment, a computer program product for generating fine resolution air pollution estimates includes a non-transitory storage medium readable by a processing circuit and storing instructions for execution by the processing circuit for performing a method. The method includes receiving historic and real-time air quality data from a plurality of air quality measurement stations in a geographic area and dividing the geographic area into a plurality of locations. The method also includes creating a composite air quality model for the geographic area based on a trend process model, a diffusion process model, a chemical process, and a containment process model based on the historic air quality data and creating a real-time air pollution estimate for each of the plurality of locations based on the composite air quality model and the real-time air quality data.

### BRIEF DESCRIPTION OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIG. 1 is a block diagram of one example of a processing system for practice of the teachings herein;

FIGS. 2A and 2B are illustrations of a geographic area having a plurality air quality measurement stations in accordance with an exemplary embodiment;

FIGS. 3A and 3B are flow diagrams of a method for modeling an air pollution containment process in accordance with an exemplary embodiment is shown; and

FIG. 4 is a flow diagram of a method for generating fine resolution air pollution estimates in accordance with an exemplary embodiment.

### DETAILED DESCRIPTION

In accordance with exemplary embodiments of the disclosure, methods, systems and computer program products for generating fine resolution air pollution estimates are provided. In exemplary embodiments, a plurality of air quality measurement stations are sparsely disposed with a geographic area and air quality data is received from each of the air quality measurement stations. The collected air quality data is input into a composite model that combines models for trend processes, diffusion processes, chemical processes, and containment processes to generate an estimate of the air quality throughout the geographic area. In exemplary embodiments, the composite model includes an estimate of air quality throughout the entire geographic area.

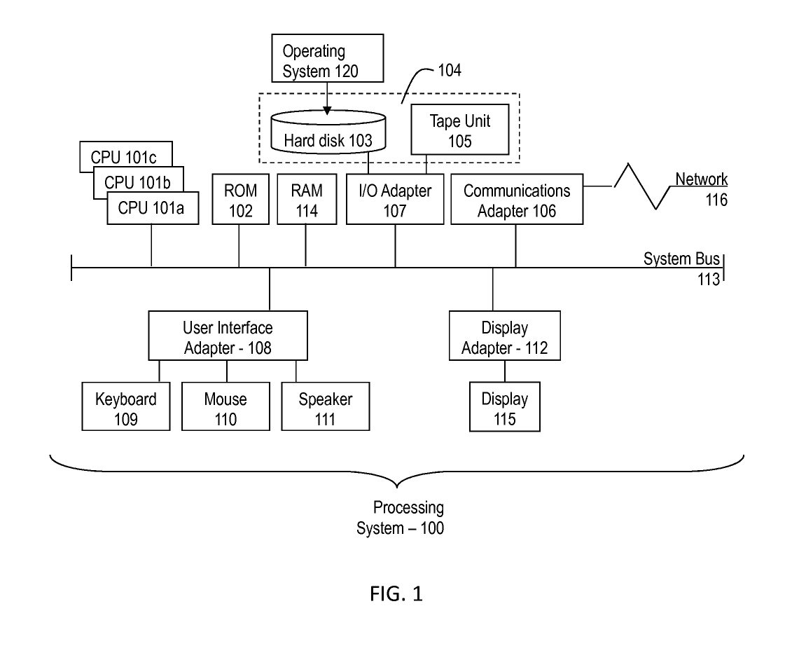

Referring now to FIG. 1, there is shown an embodiment of a processing system 100 for implementing the teachings herein. In this embodiment, the system 100 has one or more central processing units (processors) 101a, 101b, 101c, etc. (collectively or generically referred to as processor(s) 101). In one embodiment, each processor 101 may include a reduced instruction set computer (RISC) microprocessor. Processors 101 are coupled to system memory 114 and various other components via a system bus 113. Read only memory (ROM) 102 is coupled to the system bus 113 and may include a basic input/output system (BIOS), which controls certain basic functions of system 100.

FIG. 1 further depicts an input/output (I/O) adapter 107 and a network adapter 106 coupled to the system bus 113. I/O adapter 107 may be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Operating system 120 for execution on the processing system 100 may be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which may include a graphics adapter to improve the performance of graphics intensive applications and a video controller. In one embodiment, adapters 107, 106, and 112 may be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which may include, for example, a Super I/O chip integrating multiple device adapters into a single integrated circuit.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In one embodiment, a portion of system memory 114 and mass storage 104 collectively store an operating system such as the AIX® operating system from IBM Corporation to coordinate the functions of the various components shown in FIG. 1.

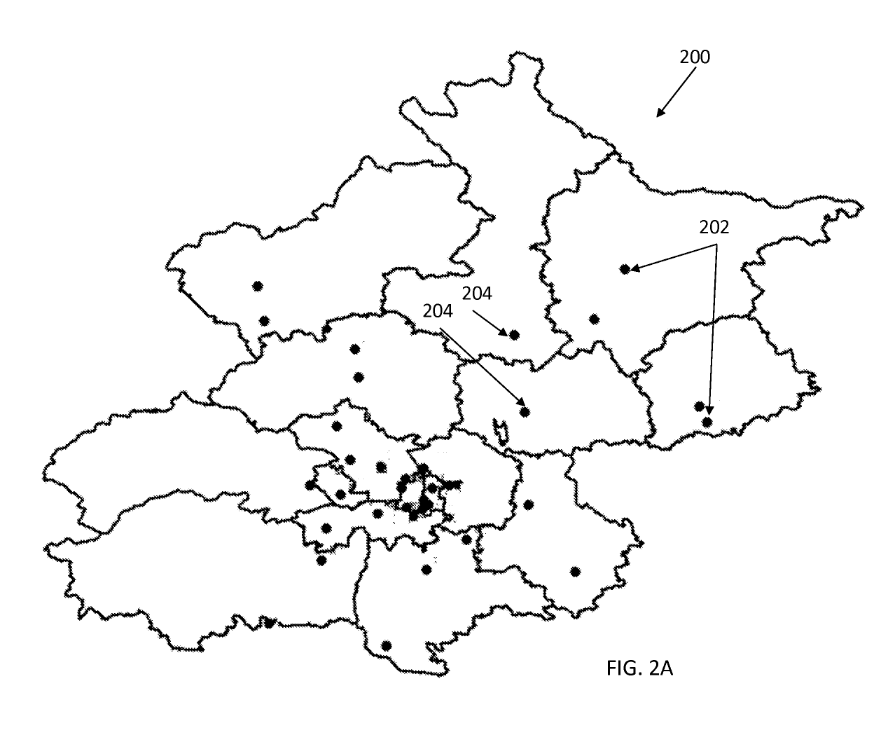

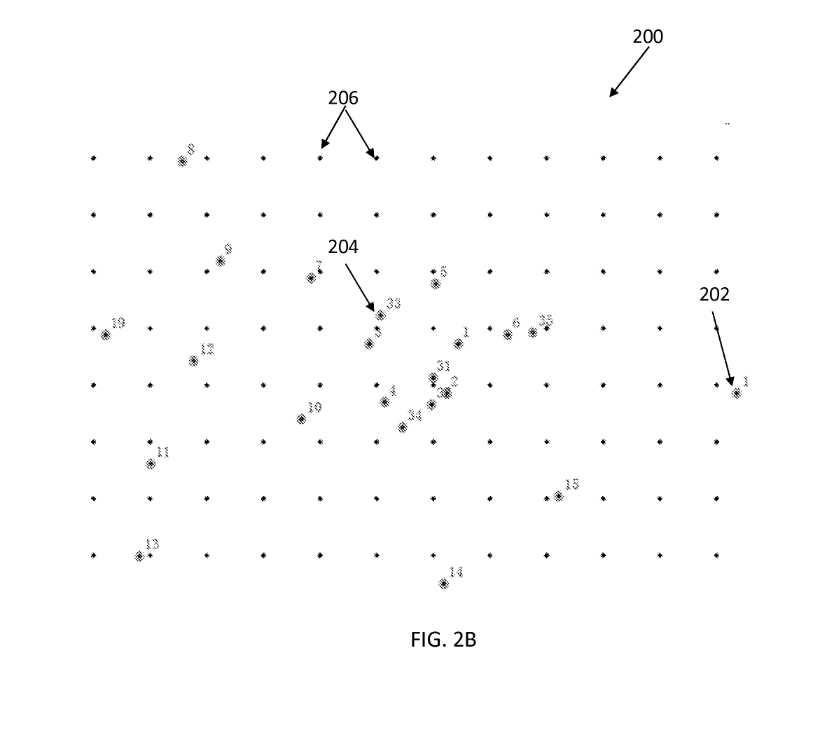

Referring now to FIGS. 2A and 2B, illustrations of a geographic area 200 having a plurality air quality measurement stations 202, 204 are shown. As illustrated, the air quality measurement stations 202, 204 are sparsely located across the geographic area. In exemplary embodiments, the air quality measurement stations 202, 204 may be uniformly or randomly dispersed in the geographic area 200. In general, the air quality measurement stations can be divided into two separate categories, edge air quality measurement stations 202 that are located near the boundary of the geographic area 200 and internal air quality measurement stations 204. In exemplary embodiments, internal air quality measurement stations 204 are located closer to one or more edge air quality measurement stations 202 than to a boundary of the geographic area 200. As shown in FIG. 2B, the geographic area 200 can be broken up into a grid of locations 206 that are evenly distributed across the geographic area 200. In exemplary embodiments, the number of locations 206 is much greater than the number of air quality measurement stations 202, 204.

In exemplary embodiments, a processing system, such as the one described above with reference to FIG. 1, is configured to receive air quality measurements from each of a plurality air quality measurement stations in a geographic area. In addition, the processing system is configured to receive weather information from a plurality of weather stations disposed across the geographic area. Furthermore, the processing system may also receive data regarding the operation of one or more pollution emission sources, such as industrial plants, vehicle congestion on highways, and the like. In exemplary embodiments, the pollution emission sources may be static sources or dynamic sources. The dynamic sources may emit pollutants in a manner that may vary in time and/or location. For example, a dynamic source may be an industrial plant that operates only on certain days or only during specific times during a day. Likewise, the dynamic sources may be emissions from motor vehicles, airplanes, trains and the like. In exemplary embodiments, a pollution model is derived from a real time analysis of the relationships between the detected levels of the air pollution and emission sources that is used to obtain the percentage of air quality variance that can be explained by the modeled emission sources. The processing system may receive air quality data and emission source data in real-time or periodically and more store the data in memory for processing.

In exemplary embodiments, the processing system is configured to create a composite model that combines models for trend processes, diffusion processes, chemical processes, and containment processes. In exemplary embodiments, the methods used for modeling trend processes, diffusion processes, chemical processes may be selected from a variety of known techniques. The method used for modeling containment processes will be described below in further detail. In exemplary embodiments, the processing system is configured to model trend processes based on dynamic environmental parameters, such as wind, rain, and the like. Likewise, the processing system is configured to model diffusion processes based on distance decay functions from known sources of diffusion pollution process sources. In addition, the processing system is configured to model chemical process based on historical and geographic data, such as the distribution of green land and waters.

In exemplary embodiments, the processing system utilizes uses data mining of historical emission patterns and historical air quality data to identify patterns, or correlations, between emission sources and the detected air quality. These patterns can be used to identify containment process parameters, which can then be incorporated into a model to generate real-time air quality for an entire geographic area based on data from the air quality monitoring stations.

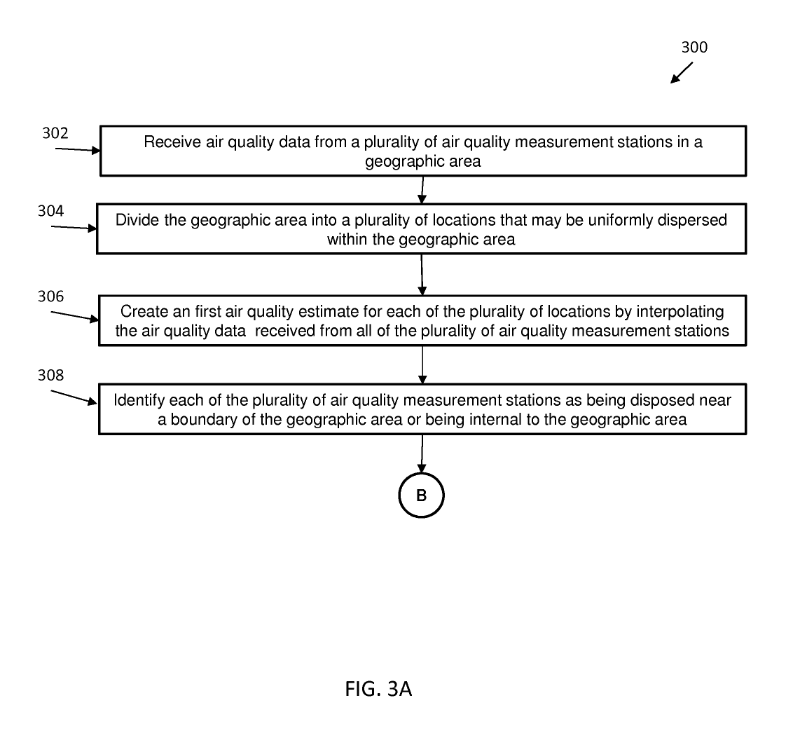

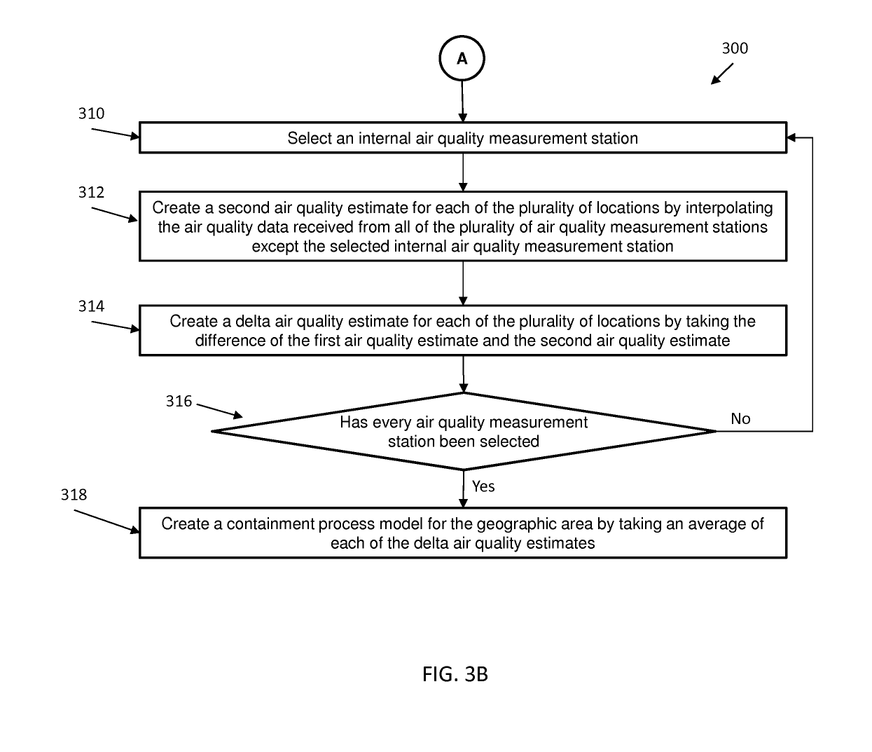

Referring now to FIG. 3A, a method 300 for modeling an air pollution containment process in accordance with an exemplary embodiment is shown. As shown at block 302, the method 300 includes receiving air quality data from a plurality of air quality measurement stations in a geographic area. In exemplary embodiments, the plurality of air quality measurement stations are sparsely dispersed in a non-uniform manner within the geographic area. Next, as shown at block 304, the method 300 includes dividing the geographic area into a plurality of locations, which may be uniformly dispersed within the geographic area. In exemplary embodiments, the number of the locations in the geographic area is considerably larger than the number of air quality measurement stations. In one embodiment, the number of the locations in the geographic area is an order of magnitude larger than the number of air quality measurement stations.

The method 300 also includes creating a first air quality estimate for each of the plurality of locations by interpolating the air quality data received from all of the plurality of air quality measurement stations, as shown at block 306. Next, as shown at block 308, the method 300 includes identifying each of the plurality of air quality measurement stations as being disposed near a boundary of the geographic area or being internal to the geographic area. Internal air quality measurement stations are located closer to one or more other air quality measurement stations than to the boundary of the geographic area.

Referring now to FIG. 3B, the method 300 for modeling air pollution containment process also includes selecting an internal air quality measurement station, as shown at block 310. Next, as shown at block 312, the method 300 includes creating a second air quality estimate for each of the plurality of locations by interpolating the air quality data received from all of the plurality of air quality measurement stations except the selected internal air quality measurement station. The method 300 also includes creating a delta air quality estimate for each of the plurality of locations by taking the difference of the first air quality estimate and the second air quality estimate, as shown at block 314. Next, as shown at decision block 316, the method 300 includes determining if every air quality measurement station been selected. If every air quality measurement station has not been selected, the method 300 returns to block 310 and selects another internal air quality measurement station. Otherwise, the method 300 proceeds to block 318 and creates a containment process model for the geographic area by taking an average of each of the delta air quality estimates created.

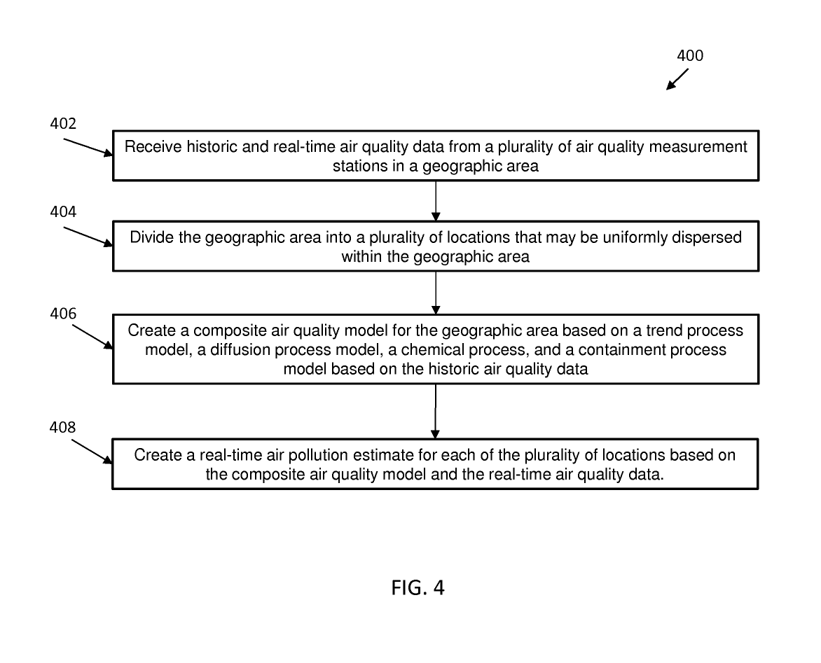

Referring now to FIG. 4, a flow diagram of a method 400 for generating fine resolution air pollution estimates in accordance with an exemplary embodiment is shown. As shown at block 402, the method 400 includes receiving historic and real-time air quality data from a plurality of air quality measurement stations in a geographic area. Next, as shown at block 404, the method 400 includes dividing the geographic area into a plurality of locations that are uniformly dispersed within the geographic area. The method 400 also includes creating a composite air quality model for the geographic area based on a trend process model, a diffusion process model, a chemical process model, and a containment process model based on the historic air quality data, as shown at block 406. In exemplary embodiments, the containment process model is created using the method shown in FIGS. 3A and 3B. Next, as shown at block 408, the method 400 includes creating a real-time air pollution estimate for each of the plurality of locations based on the composite air quality model and the real-time air quality data.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for generating fine resolution air pollution estimates, the method comprising:
receiving historic and real-time air quality data from a plurality of air quality measurement stations in a geographic area;
dividing the geographic area into a plurality of locations;
creating, by a processor, a composite air quality model for the geographic area based on a trend process model, a diffusion process model, a chemical process, and a containment process model based on the historic air quality data; and
creating, by the processor, a real-time air pollution estimate for each of the plurality of locations based on the composite air quality model and the real-time air quality data.

2. The method of claim 1, wherein the plurality of air quality measurement stations are sparsely and non-uniformly disposed in the geographic area.

3. The method of claim 2, wherein the plurality of location are uniformly dispersed within the geographic area.

4. The method of claim 3, wherein a number of the plurality of location is an order of magnitude larger than a number of the plurality of air quality measurement stations.

5. The method of claim 1, wherein the containment process model is created by:
creating a first air quality estimate for each of the plurality of locations by interpolating the historic air quality data received from all of the plurality of air quality measurement stations; and
identifying each of the plurality of air quality measurement stations an internal air quality measurement stations or a boundary air quality measurement stations.

6. The method of claim 5, wherein creating the containment process model further comprises:
iteratively selecting each of the internal air quality measurement stations;
creating second air quality estimates for each of the plurality of locations by interpolating the historic air quality data received from all of the plurality of air quality measurement stations except the selected internal air quality measurement station;
creating delta air quality estimates for each of the plurality of locations by taking the difference of the first air quality estimate and the second air quality estimate; and
creating the containment process model for the geographic area by taking an average of each of the delta air quality estimates created.

7. The method of claim 5, wherein the internal air quality measurement stations are located closer to one or more other air quality measurement stations than to a boundary of the geographic area.
